(19) United States
(12) Patent Application Publication
(10) Pub. No.: US 2021/0386738 A1
(43) Pub. Date: Dec. 16, 2021

(54) COMBINATION OF PRMT5 INHIBITORS AND BCL-2 INHIBITORS

(71) Applicant: GLAXOSMITHKLINE INTELLECTUAL PROPERTY DEVELOPMENT LIMITED, Brentford, Middlesex (GB)

(72) Inventor: Olena BARBASH, Collegeville, PA (US)

(21) Appl. No.: 17/291,793

(22) PCT Filed: Nov. 6, 2019
(86) PCT No.: PCT/EP2019/080386

§ 371 Date: May 6, 2021

Related U.S. Application Data
(60) Provisional application No. 62/757,352, filed on Nov. 8, 2018.

(51) Int. Cl. A61K 31/506; A61K 31/4725; A61K 31/635; A61K 31/5377; A61K 31/336; A61K 31/352; A61K 31/426; A61K 31/05; A61K 31/167; A61K 31/404; A61P 35/00

(52) U.S. Cl. CPC A61K 31/506; A61K 31/4725; A61K 31/635; A61K 31/5377; A61K 31/336; A61P 35/00; A61K 31/426; A61K 31/05; A61K 31/167; A61K 31/404; A61K 31/352

(57) ABSTRACT

The present invention relates to a combination of a protein arginine methyltransferase 5 (PRMT5) inhibitor and a B cell lymphoma 2 (BCL-2) inhibitor and the use of this combination in the treatment of cancer. In specific instances of the invention the PRMT5 inhibitor may be a compound of Formula (I).

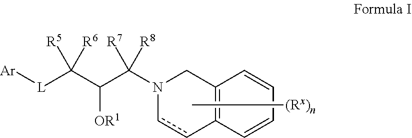

### FIELD OF THE INVENTION

The present invention relates to a method of treating cancer and combinations useful in such treatment. In particular, the present invention relates to a combination of a protein arginine methyltransferase 5 (PRMT5) inhibitor and a B cell lymphoma 2 (BCL-2) inhibitor.

### BACKGROUND TO THE INVENTION

Effective treatment of hyperproliferative disorders, including cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Increasing evidence suggests that Protein Arginine Methyltransferase 5 (PRMT5) is involved in tumorigenesis. PRMT5 protein is overexpressed in a number of cancer types, including lymphoma, glioma, breast and lung cancer. Knockdown of PRMT5 often leads to a decrease in cell growth and survival in cancer cell lines. The strongest mechanistic link currently described between PRMT5 and cancer is in mantle cell lymphoma (MCL). Recent data suggest that PRMT5 inhibition could be used as a therapeutic strategy in MCL. Many PRMT5 inhibitors have been discovered in the past five years and some have more recently entered clinical trials for the treatment of solid tumours and non-Hodgkin lymphoma (specifically MCL).

BCL-2 is another protein which has been shown to be associated with tumour initiation, tumour progression and lack of response to chemotherapy in many cancers. It has previously been shown that inhibition of BCL-2 gives promising results in haematological malignancies. Venetoclax, also known as ABT-199, (a BCL-2 inhibitor) has recently been approved by the FDA for patients with chronic lymphocytic leukemia (CLL) or small lymphocytic lymphoma (SLL), who have received at least one prior therapy.

Although there have been many recent advances in the treatment of cancer, there remains a need in the art for more effective and/or enhanced treatment of individuals suffering the effects of cancer.

### SUMMARY OF THE INVENTION

The present invention provides a combination of a PRMT5 inhibitor and a BCL-2 inhibitor.

In another aspect, the present invention provides a method of treating cancer in a human in need thereof, the method comprising administering to the human a combination as defined herein, thereby treating the cancer in the human.

In another aspect, the present invention also provides a combination as defined herein for use in the treatment of cancer.

In a further aspect, the present invention provides a pharmaceutical composition comprising the combination defined herein and a pharmaceutically acceptable carrier.

Also provided is a kit comprising a PRMT5 inhibitor and BCL-2 inhibitor as defined herein.

The present invention is advantageous in a number of respects. Specifically, the combination of the present invention may have a synergistic effect when used to treat a patient with cancer.

### BRIEF DESCRIPTION OF THE DRAWINGS

FIG. 1 shows a work flow for the cell growth/cell death assay used in the examples of the present invention.

FIG. 2 shows titrations used for serial dilutions with ABT-199, 16 points from row A in combination with PRMT5 inhibitor (compound A).

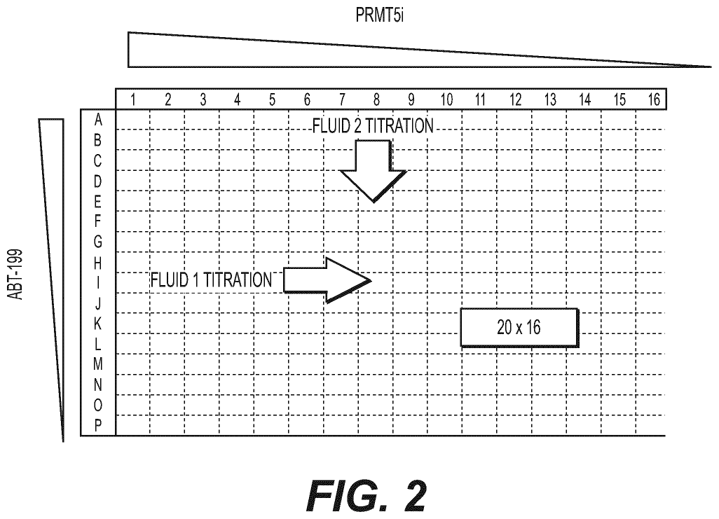

FIG. 3 shows a curve showing the percentage of T=0 (a percentage of the T₀ value (value at time=0). The T₀ value was normalized to 100% and represents the number of cells present at the time of compound addition. Results were background corrected by subtraction of values from wells containing no cells) against concentration (nM) in the lymphoma cell line DB treated with ABT-199 and Compound A as single agents and in combination.

FIG. 4 shows a curve showing the percentage of T=0 against concentration (nM) in the lymphoma cell line SU-DHL-4 treated with ABT-199 and Compound A as single agents and in combination.

FIG. 5 shows a curve showing the percentage of T=0 against concentration (nM) in the lymphoma cell line U-2932 treated with ABT-199 and Compound A as single agents and in combination.

FIG. 6 shows a curve showing the percentage of T=0 against concentration (nM) in the lymphoma cell line WSU-FSCCL treated with ABT-199 and Compound A as single agents and in combination.

FIG. 7 shows a curve showing the percentage of T=0 against concentration (nM) in the lymphoma cell line OCI-LY3 treated with ABT-199 and Compound A as single agents and in combination.

### DESCRIPTION OF VARIOUS EMBODIMENTS

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centres, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et ah, Enantiomers, Racemates and Resolutions (Wiley Interscience, New York, 1981); Wilen et ah, Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “aliphatic,” as used herein, includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

When a range of values is listed, it is intended to encompass each value and subrange within the range. For example, “C₁₋₆ alkyl” is intended to encompass, C₁; C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “radical” as used herein refers to a point of attachment on a particular group. Radical includes divalent radicals of a particular group.

The term “alkyl”, as used herein, refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g. unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

The term “alkenyl” as used herein, refers to a radical of a straight-chain or branched hydrocarbon group having from 2 to 20 carbon atoms and one or more carbon-carbon double bonds (e.g., 1, 2, 3, or 4 double bonds), and optionally one or more triple bonds (e.g., 1, 2, 3, or 4 triple bonds) (“C₂₋₂₀ alkenyl”). In certain embodiments, alkenyl does not comprise triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”) In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-4 alkenyl groups include ethenyl (C₂), 1-propenyl (C₃), 2-propenyl (C₃), 1-butenyl (C₄), 2-butenyl (C₄), butadienyl (C₄), and the like. Examples of C₂-6 alkenyl groups include the aforementioned C₂-4 alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g. unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

The term “alkynyl” as used herein refers to a radical of a straight-chain or branched hydrocarbon group having from 2 to 20 carbon atoms and one or more carbon-carbon triple bonds (e.g., 1, 2, 3, or 4 triple bonds), and optionally one or more double bonds (e.g., 1, 2, 3, or 4 double bonds) (“C₂₋₂₀ alkynyl”). In certain embodiments, alkynyl does not comprise double bonds. Examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

The terms “carbocyclyl” or “carbocyclic”, as used herein, refers to a radical of a non-aromatic cyclic hydrocarbon group having from 3 to 14 ring carbon atoms (“C₃₋₁₄ carbocyclyl”) and zero heteroatoms in the non-aromatic ring system. Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, the carbocyclyl group may be monocyclic or is a fused, bridged or spiro-fused ring system such as a bicyclic system and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents.

The term “Heterocyclyl” or “heterocyclic” as used herein refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g. unsubstituted (an “unsubstituted aryl”) or substituted (a “substituted aryl”) with one or more substituents.

As used herein, the term “heteroaryl” refers to a radical of a 5-14 membered monocyclic or polycyclic (e.g., bicyclic or tricyclic) 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, aliphatic, alkyl, alkenyl, alkynyl, carbocyclyl, heterocyclyl, aryl, and heteroaryl groups, as defined herein, are optionally substituted e.g., “substituted” or “unsubstituted”. In general, as used herein, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

As used herein, the term “halo” or “halogen” refers to fluorine (fluoro, —F), chlorine (chloro, —Cl), bromine (bromo, —Br), or iodine (iodo, —I).

As used herein, the term “pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66: 1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, quaternary salts.

### Combinations

In a first aspect, the present invention provides a combination of a PRMT5 inhibitor and a BCL-2 inhibitor.

The PRMT5 inhibitor used in the combination of the present invention may be any PRMT5 inhibitor known in the art. In a specific embodiment of the invention the PRMT5 inhibitor is a compound of Formula I:

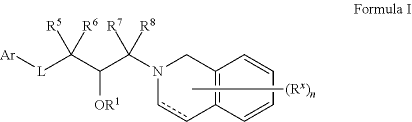

or a pharmaceutically acceptable salt thereof,

wherein
[symbol] : represents a single or double bond;
R¹ is hydrogen, R^{z}, or —C(O)R^{z}, wherein R^{z} is optionally substituted C₁₋₆ alkyl;
L is —N(R)C(O)—, —C(O)N(R)—, —N(R)C(O)N(R)—, —N(R)C(O)O—, or —OC(O)N(R)—;
each R is independently hydrogen or optionally substituted C₁₋₆ aliphatic;
Ar is a monocyclic or bicyclic aromatic ring having 0-4 heteroatoms independently selected from nitrogen, oxygen, and sulfur, wherein Ar is substituted with 0, 1, 2, 3, 4, or 5 R^{y} groups, as valency permits;
each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A} N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NRBC(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;
each R^{A} is independently selected from the group consisting of hydrogen, optionally substituted aliphatic, optionally substituted carbocyclyl, optionally substituted heterocyclyl, optionally substituted aryl, and optionally substituted heteroaryl;
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;
R⁵, R⁶, R⁷, and R⁸ are independently hydrogen, halo, or optionally substituted aliphatic;
each R^{X} is independently selected from the group consisting of halo, —CN, optionally substituted aliphatic, —OR′, and —N(R″)₂;
R′ is hydrogen or optionally substituted aliphatic;
each R″ is independently hydrogen or optionally substituted aliphatic, or two R″ are taken together with their intervening atoms to form a heterocyclic ring; and
n is 0, 1, 2, 3, 4, 5, 6, 7, 8, 9, or 10, as valency permits.

In one embodiment the compound of Formula I is a compound of Formula Ia or a pharmaceutically acceptable salt thereof.

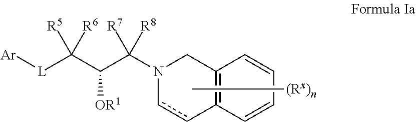

In another embodiment, the compound of Formula I is a compound of Formula Ib or a pharmaceutically acceptable salt thereof.

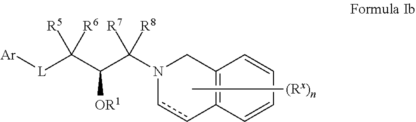

In an embodiment of the invention, L is —N(R)C(O)—. In an embodiment of the invention L is —N(R)C(O)— and R is H. In another embodiment of the invention n is 0. In an embodiment of the invention L is —N(R)C(O)—, n is 0 and R is H.

In an embodiment of the invention, the PRMT5 inhibitor is a compound of Formula II

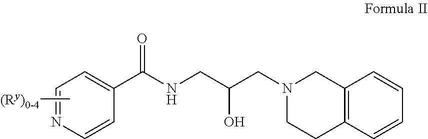

or a pharmaceutically acceptable salt thereof.

In an embodiment of the invention R^{y} is —NHR^{B}. In an embodiment R^{B} is an optionally substituted carbocyclyl.

In an embodiment of the invention, the PRMT5 inhibitor is Compound A, or a pharmaceutically acceptable salt thereof.

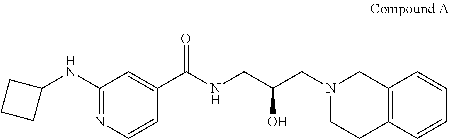 [SMILES: O=C(NC[C@H](O)CN1CCc2ccccc2C1)c1ccnc(NC2CCC2)c1]

Compound A and methods of making compound A are disclosed in PCT/US2013/077235, on at least page 139 (Compound 188).

In one embodiment, the PRMT5 inhibitor is a compound of Formula III:

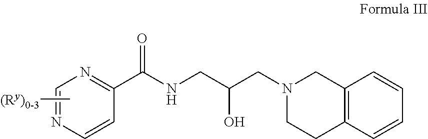

or a pharmaceutically acceptable salt thereof.

In one embodiment, the PRMT5 inhibitor is a compound of Formula IV:

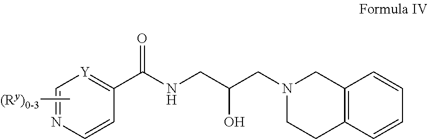

or a pharmaceutically acceptable salt thereof, wherein Y is CH or N.

In one embodiment, R^{y} is —NHR^{B}. In another embodiment, R^{B} is optionally substituted heterocyclyl. In an embodiment R^{y} is —NHR^{B} and R^{B} is an optionally substituted heterocyclyl.

In an embodiment, the PRMT5 inhibitor is a compound of Formula IX:

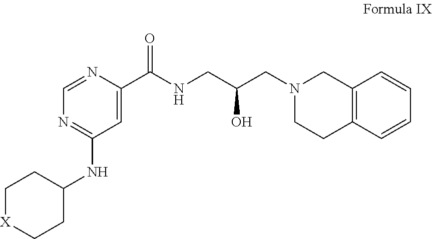

or a pharmaceutically acceptable salt thereof, wherein X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl.

In one embodiment, the PRMT5 inhibitor is Compound B:

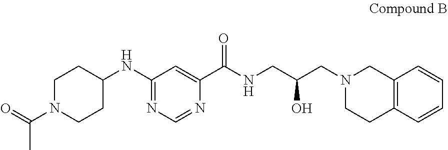 [SMILES: CC(=O)N1CCC(Nc2cc(C(=O)NC[C@H](O)CN3CCc4ccccc4C3)ncn2)CC1]

or a pharmaceutically acceptable salt thereof. Compound B and methods of making Compound B are disclosed in PCT/US2013/077235, in at least page 141 (Compound 208) and page 291, paragraph [00464] to page 294, paragraph [00469].

PRMT5 inhibitors are further disclosed in PCT/US2013/077235 and PCT/US2015/043679, which are incorporated herein by reference. Exemplary Type II PRMT inhibitors are disclosed in Table 1A, Table 1B, Table 1C, Table 1D, Table 1E, Table 1F, and Table 1G of PCT/US2013/077235, and methods of making the Type II PRMT inhibitors are described in at least page 239, paragraph [00359] to page 301, paragraph [00485] of PCT/US2013/077235. The generic and specific compounds described in these patent applications are incorporated herein by reference and can be used to treat cancer as described herein.

In an embodiment of the invention the BCL-2 inhibitor may be any suitable BCL-2 inhibitor known to a person of skill in the art. There are many known BCL-2 inhibitors including a number in clinical trials.

In an embodiment of the invention the BLC-2 inhibitor is venetoclax (ABT-199), ABT-737, navitoclax (ABT-263), APG-1252, S-055746, BDA-366, HA14-1, BH3I-1, apogossypol, TW-37, TM12-06 or obatoclax. In an embodiment of the invention, the BLC2 inhibitor is venetoclax, ABT-737, navitoclax, APG1252, obatoclax. In a further embodiment of the invention, the BCL-2 inhibitor is venetoclax.

### Methods of Treatment

In a second aspect, the present invention provides a method of treating cancer in a human in need thereof, the method comprising administering to the human a combination as defined herein, thereby treating the cancer in the human.

In an embodiment the method comprises administering to the human a combination as defined herein, together with at least one of: a pharmaceutically acceptable carrier and a pharmaceutically acceptable diluent, thereby treating the cancer in the human.

In a further aspect of the invention, a pharmaceutical composition comprising a combination as defined herein is provided. In an embodiment, the pharmaceutical composition comprises a therapeutically active amount of the PRMT5 inhibitor and a therapeutically active amount of the BCL-2 inhibitor.

In a still further aspect, the present invention provides a method of treating cancer in a human in need thereof, the method comprising administering to the human a pharmaceutical composition as defined herein, thereby treating the cancer in the human.

In an embodiment the method comprises administering to the human the pharmaceutical composition as defined herein, together with at least one of: a pharmaceutically acceptable carrier and a pharmaceutically acceptable diluent, thereby treating the cancer in the human.

In a still further aspect of the present invention, there is provided a combination, as defined herein, for use in therapy.

In a still further aspect of the present invention, there is provided, a combination, as defined herein, for use in the treatment of cancer.

In the aspects discussed above, the cancer is a solid tumor or a haematological cancer. In an embodiment the cancer is lung cancer or lymphoma.

In one embodiment the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

In an embodiment the cancer is lymphoma. In an embodiment the cancer is malignant lymphoma, hodgkins lymphoma, non-hodgkins lymphoma, lymphoblastic T cell lymphoma, Burkitt's lymphoma, diffuse large B cell lymphoma (DLBCL) or follicular lymphoma. In an embodiment, the cancer is diffuse large B cell lymphoma (DLBCL).

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer” and “tumour” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large B cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In any one of the aspects discussed above, the PRMT5 inhibitor and the BCL-2 inhibitor may be administered together in a single pharmaceutical composition or separately and, when administered separately this may occur simultaneously or sequentially in any order.

Suitably, the combinations of this invention are administered within a “specified period”.

The term “specified period” and grammatical variations thereof, as used herein, means the interval of time between the administration of one of a PRMT5 inhibitor and a BCL2 inhibitor and the other of a PRMT5 inhibitor and a BCL2 inhibitor. Unless otherwise defined, the specified period can include simultaneous administration. Unless otherwise defined, the specified period refers to administration of a PRMT5 inhibitor and a BCL2 inhibitor during a single day.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of a PRMT5 inhibitor and a BCL2 inhibitor in less than about 45 minutes apart is considered simultaneous administration.

Suitably, when the combination of the invention is administered for a “specified period”, the compounds will be co-administered for a “duration of time”.

The term “duration of time” and grammatical variations thereof, as used herein means that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

Regarding “specified period” administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and grammatical derivates thereof, as used herein is meant that one of a PRMT5 inhibitor and a BCL2 inhibitor is administered once a day for two or more consecutive days and the other of a PRMT5 inhibitor and a BCL2 inhibitor is subsequently administered once a day for two or more consecutive days. Also, contemplated herein is a drug holiday utilized between the sequential administration of one of a PRMT5 inhibitor and A BCL2 inhibitor and the other of PRMT5 inhibitor and a BCL2 inhibitor. As used herein, a drug holiday is a period of days after the sequential administration of one of a PRMT5 inhibitor and a BCL2 inhibitor and before the administration of the other of a PRMT5 inhibitor and a BCL2 inhibitor where neither the PRMT5 inhibitor nor the BCL2 inhibitor is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

### Regarding Sequential Administration:

Suitably, one of a PRMT5 inhibitor and a BCL2 inhibitor is administered for from 1 to 30 consecutive days, followed by an optional drug holiday, followed by administration of the other of the PRMT5 inhibitor and the BCL2 inhibitor for from 1 to 30 consecutive days. Suitably, one of a PRMT5 inhibitor and a BCL2 inhibitor is administered for from 1 to 21 consecutive days, followed by an optional drug holiday, followed by administration of the other of the PRMT5 inhibitor and the BCL2 inhibitor for from 1 to 21 consecutive days. Suitably, one of a PRMT5 inhibitor and a BCL2 inhibitor is administered for from 1 to 14 consecutive days, followed by a drug holiday of from 1 to 14 days, followed by administration of the other of the PRMT5 inhibitor and the BCL2 inhibitor for from 1 to 14 consecutive days. Suitably, one of a PRMT5 inhibitor and a BCL2 inhibitor is administered for from 1 to 7 consecutive days, followed by a drug holiday of from 1 to 10 days, followed by administration of the other of the PRMT5 inhibitor and the BCL2 inhibitor for from 1 to 7 consecutive days.

It is understood that a “specified period” administration and a “sequential” administration can be followed by repeat dosing or can be followed by an alternate dosing protocol, and a drug holiday may precede the repeat dosing or alternate dosing protocol.

The PRMT5 inhibitor and the BCL2 inhibitor of the invention may be administered by any appropriate route. Suitable routes include oral, rectal, nasal, topical (including buccal and sublingual), intratumorally, vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the PRMT5 inhibitor and the BCL2 inhibitor may be compounded together in a pharmaceutical composition/formulation.

The combination of the present invention may also be employed with other therapeutic methods of cancer treatment i.e. the combination defined herein may be administered to a subject who is already receiving treatment for a cancer. The subject may be receiving, for example, chemotherapy.

The combination of the present invention may be administered by any suitable route.

In a final aspect, the present invention provides a kit comprising a protein arginine methyltransferase 5 (PRMT5) inhibitor as defined herein and a B cell lymphoma 2 (BCL-2) inhibitor as defined herein.

The following non-limiting Examples illustrate the present invention.

### Examples

The activity of compound A (a PRMT5 inhibitor) with therapeutic agent ABT-199 (a BCL-2 inhibitor) was determined in various cell lines.

### Cell Culture

The majority cell lines were obtained from GSK BioCat group, American Type Culture Collection (ATCC), or the Deutsche Sammlung von Mikroorganismen and Zellbulturen (DSMZ). All cell lines were maintained in growth medium in T75 cm² flasks, incubated at 37° C. with 5% CO₂ and split every 3 to 4 days. Growth media conditions and cell line information are detailed in Table 1. Cells were counted using the Vi-Cell Analyzer (Beckman Coulter ViCell) to obtain viable cell counts.

TABLE 1
Growth media conditions and cell line information (FBS:
Fetal bovine serum, RPMI-1640: Roswell Park Memorial Institute
1640 medium, IMDM: Iscove's Modified Dulbecco's Medium)

| Cell line | Medium |
|---|---|
| DB | RPMI-1640 + 20% FBS, 1% Glutamax, 1% sodium |
| | pyruvate |
| DOHH-2 | RPMI-1640 + 20% FBS, 1% Glutamax, 1% sodium |
| | pyruvate |
| HT | RPMI-1640 + 10% FBS, 1% Glutamax, 1% sodium |
| | pyruvate |
| JVM2* | RPMI-1640 + 10% FBS, 1% Glutamax, 1% sodium |
| | pyruvate |
| NALM-6 | RPMI-1640 + 10% FBS, 1% Glutamax, 1% sodium |
| | pyruvate |
| NALM-6 | RPMI-1640 + 10% FBS, 1% Glutamax, 1% sodium |
| (B-ALL) | pyruvate |
| NU-DUL-1 | RPMI-1640 + 15% FBS, 1% Glutamax, 1% sodium |
| | pyruvate |
| OCI-Ly10 | IMDM + 20% FBS |
| OCI-LY3 | IMDM + 20% FBS |
| RC-K8 | RPMI-1640 + 15% FBS, 1% Glutamax, 1% sodium |
| | pyruvate |
| RIVA | RPMI-1640 + 20% FBS, 1% Glutamax, 1% sodium |
| | pyruvate |
| RL | RPMI-1640 + 20% FBS, 1% Glutamax, 1% sodium |
| | pyruvate |
| SC1 | RPMI-1640 + 20% FBS, 1% Glutamax, 1% sodium |
| | pyruvate |
| SU-DHL-10 | RPMI-1640 + 20% FBS, 1% Glutamax, 1% sodium |
| | pyruvate |
| SU-DHL-2 | RPMI-1640 + 10% FBS, 1% Glutamax, 1% sodium |
| | pyruvate |
| SU-DHL-4 | RPMI-1640 + 20% FBS, 1% Glutamax, 1% sodium |
| | pyruvate |
| SU-DHL-5 | RPMI-1640 + 20% FBS, 1% Glutamax, 1% sodium |
| | pyruvate |
| SU-DHL-6 | RPMI-1640 + 20% FBS, 1% Glutamax, 1% sodium |
| | pyruvate |
| SU-DHL-8 | RPMI-1640 + 10% FBS, 1% Glutamax, 1% sodium |
| | pyruvate |
| Toledo | RPMI-1640 + 20% FBS, 1% Glutamax, 1% sodium |
| | pyruvate |
| U-2932 | RPMI-1640 + 20% FBS, 1% Glutamax, 1% sodium |
| | pyruvate |
| U-2940 | RPMI-1640 + 20% FBS, 1% Glutamax, 1% sodium |
| | pyruvate |
| WSU-DLCL2 | RPMI-1640 + 20% FBS, 1% Glutamax, 1% sodium |
| | pyruvate |
| WSU-FSCCL | RPMI-1640 + 20% FBS, 1% Glutamax, 1% sodium |
| | pyruvate |
| WSU-NHL | RPMI-1640 + 20% FBS, 1% Glutamax, 1% sodium |
| | pyruvate |
| Z-138 | IMDM + 10% HI Horse serum |

### Materials

Compound A was provided by known methods. Commercially available ABT-199 was quality checked before use.

Gamma irradiated, heat inactivated FBS (cat. #12176C-1000ML was purchased from SAFC Biosciences. HI Horse serum (Gibco, cat: 26050-088), cell culture base medium RPMI-1640 (Gibco, cat: A22400-071), IMDM (Gibco, cat: 12440-046), GlutaMAX™-I (100X) (cat. #35050-061) and Sodium Pyruvate (100 mM) (cat. 11360-070) were all purchased from Gibco/Invitrogen, Carlsbad, Calif.

CellTiter-Glo™ luminescent cell viability assay reagent was purchased from Promega Corp., Madison, Wis. (cat. #G8462).

### Standard 6-Day Growth-Death Assay

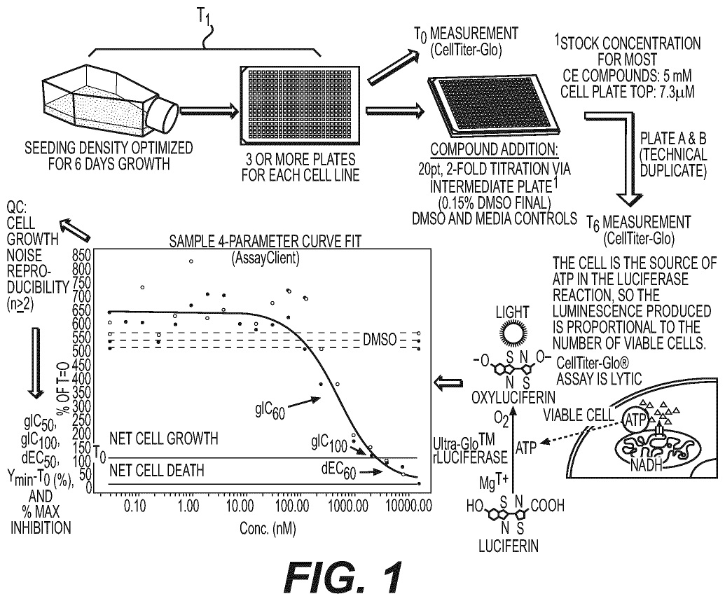

Cell proliferation assays were performed on a panel of cell lines according to the work flow shown in FIG. 1. Optimal cell seeding density was determined for all cell lines by monitoring proliferation over a range of seeding densities in 384-well format and identifying the seeding density at which cells grew logarithmically throughout six days. 50 uL of cells per well were plated in 384-well plates at the optimal seeding density in preferred supplemented culture media. Three plates were prepared per cell line and incubated overnight at 37° C. in 5% CO₂.

In these studies, lymphoma cell lines were treated with PRMT5 inhibitor, compound A and BCL-2 targeted inhibitor, ABT-199 single agent or in combination using a fixed 1:1 ratio of each inhibitor over a 20-point titration. For compound A single and fixed 1:1 ratio combination with ABT-199 cell proliferation assay, cells were treated in duplicate with a 20-point, two-fold dilution series of Compound A, ABT-199 (≥14 μM top dose) single or combo treatment, and ≤0.15% DMSO final concentration. Plates were incubated for six days at the conditions described above. Cell growth was measured using CellTiter-Glo (Promega) and luminescence signal was detected with a BioTek Synergy Neo microplate reader. A plate of untreated cells was read at the time of compound/biologic addition to determine the T₀ value representing the starting number of cells. Data were fit with a four-parameter equation to generate a concentration response curve using Assay Client software.

### Data Analysis

Results were background corrected by subtraction of values from wells containing no cells, expressed as a percentage of the T₀ value (value at time=0), and plotted against compound concentration. The T₀ value was normalized to 100% and represents the number of cells present at the time of compound addition. The cellular response was determined for each compound using a 4- or 6-parameter curve fit of cell viability against concentration using Assay Client software. Growth IC50 (gIC50) values correspond to the concentration intersecting the mid-point of the growth window (between DMSO and T0 values). Growth IC₁₀₀ (gIC₁₀₀) values correspond to the concentration at which 100% growth inhibition is achieved, based on the growth window. The Ymin−T₀ value, a measure of net population cell growth or death, is calculated by subtracting the T₀ value (100%) from the Ymin value (%) that is determined from the fit of the concentration response curve. Death EC50 (dEC50) values correspond to the concentration at which 50% net cell death is observed.

Individual curves were quality checked for three criteria: outlier data points, cell growth (population doubling ≥1.5), and proper curve fitting. Curves were omitted from the analysis if they did not meet the above criteria. Individual curves were QC checked for outlier data points, cell growth (population doubling ≥1.5), and proper curve fitting. Obvious outlier data points were omitted only as needed to ensure proper curve fit. Full dose response curves were omitted as indicated “QC” in the results table if they did not meet these criteria.

For fixed ratio screening, combination activity was assessed by the fold change in gIC₅₀ compared to the 2 single agent curves by dividing the compound ratio as necessary. Combinations showing >3-fold and >5-fold change from single agent are described as below:
Fold changes ≥5 are considered to represent strong synergy.
Fold changes between 3.0 and 4.9 are considered to represent weak synergy.
Fold changes <3 are considered to represent no synergy.

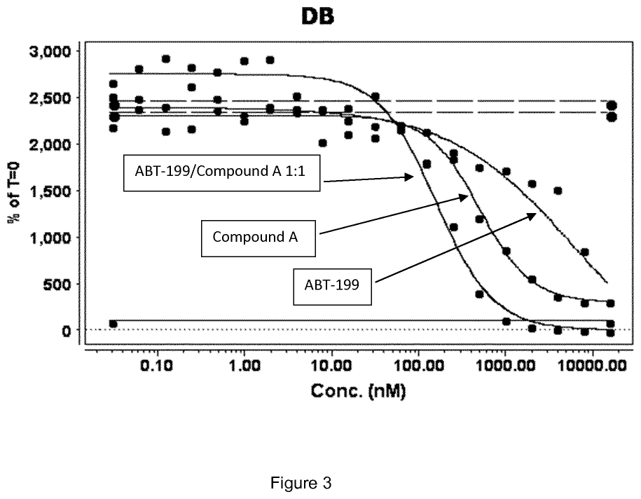

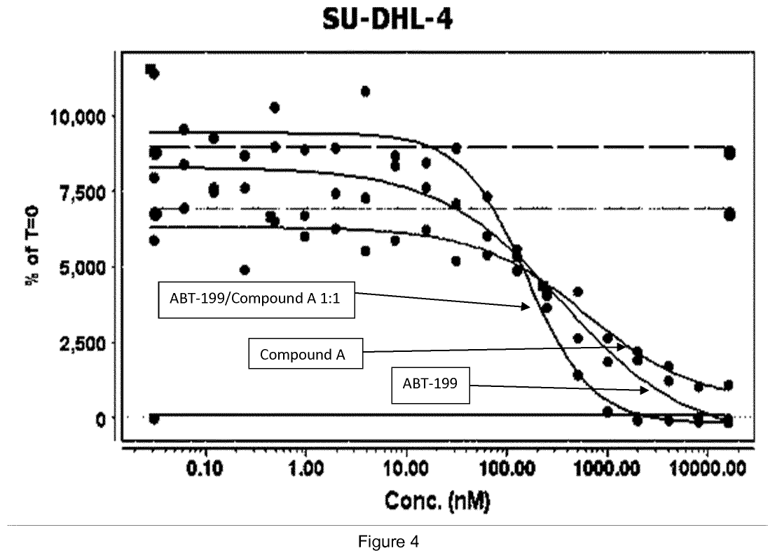

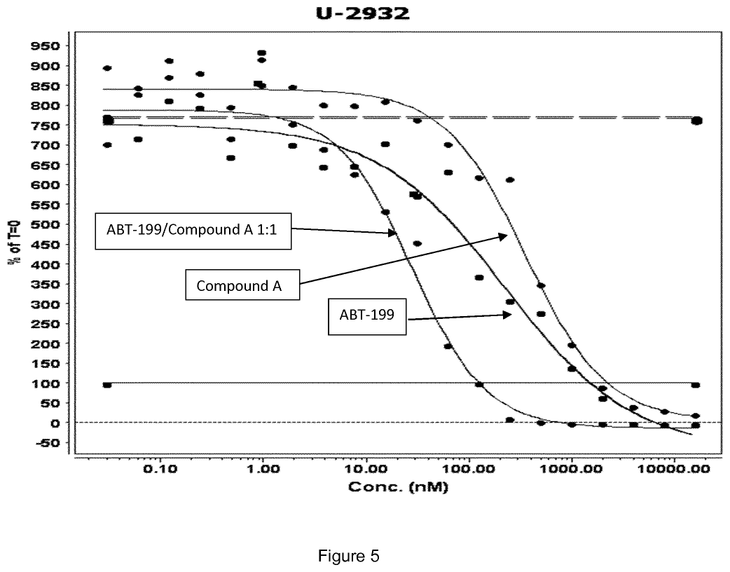

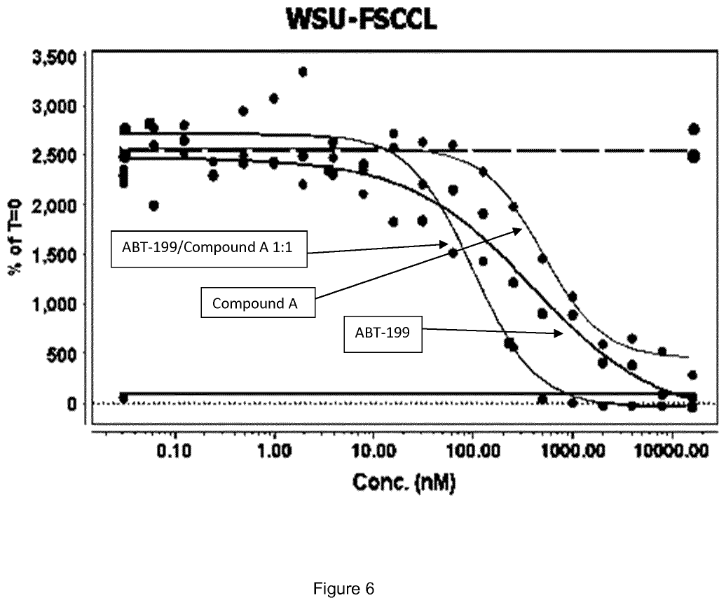

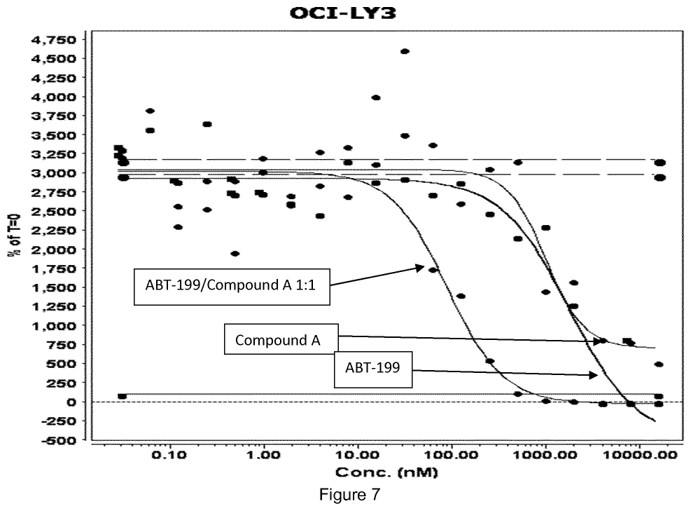

FIGS. 3-7 are curves comparing concentration (nM) versus % of T=0 for single ABT-199, single compound A and the combination in five of the tested cell lines. In each of the cell lines depicted, the combination causes greater cell death at a lower concentration thus showing synergy.

The combination showed strong synergy in a subset of cell lines (11/25 cell lines relative to either single agent). The combination resulted in in ≥3-fold more potent gIC₅₀ in 7/25 cell lines relative to either single agent, with a ≥5-fold increase in 2 cell lines. Where gIC₅₀ is the midpoint of the ‘growth window’, the difference between the number of cells at the time of compound addition (T₀) and the number of cells after 6 days (DMSO control). The combination treatment resulted in a ≥5 fold shift in gIC₁₀₀ in 6 cell lines and ≥10 fold shift in gIC₁₀₀ in 3 cell lines. Where a gIC₁₀₀ value represents the concentration of compound required for 100% inhibition of growth. The combination also resulted in a ≥5 fold shift in dEC₅₀ in 6 cell lines and ≥10 fold shift in dEC₅₀ in 3 cell lines. Where dEC₅₀ is death EC50 i.e. the concentration at which 50% net cell death is observed.

These data indicate that a profound combination effect on inhibition of growth can be achieved through the simultaneous inhibition of PRMT5 and BCL-2. Tables 2a-2j show Six-day fixed ratio combination treatment in lymphoma lines. gIC50, gIC100, dEC50 [nM] for single and combination treatments, and fold change over single agents (average of n=2). Table 3 shows a summary of the fold change from the most potent agent.

TABLE 2a

| | gIC50 | gIC100 | dEC50 | Ymin-T0 |
|---|---|---|---|---|
| DB | (nM) | (nM) | (nM) | (%) |
| COMPOUND A | 862 | 14663 | 14663 | 194 |
| ABT-199 | 2482 | 14663 | 14663 | 449 |
| ABT99/GSK591_1/1 | 221 | 2349 | 3472 | −96 |
| fold change from PRMT5i | 3.9 | 6.2 | 4.2 | |
| fold change from BCL-2i | 11.2 | 6.2 | 4.2 | |

TABLE 2b

| | gIC50 | gIC100 | dEC50 | Ymin-T0 |
|---|---|---|---|---|
| DOHH-2 | (nM) | (nM) | (nM) | (%) |
| COMPOUND A | 41 | 1474 | 1937 | −100 |
| ABT-199 | 51 | 2897 | 1818 | −69 |
| ABT99/GSK591_1/1 | 21 | 290 | 387 | −100 |
| fold change from PRMT5i | 2.0 | 5.1 | 5.0 | |
| fold change from BCL-2i | 2.5 | 10.0 | 4.7 | |

TABLE 2c

| | gIC50 | gIC100 | dEC50 | Ymin-T0 |
|---|---|---|---|---|
| SU-DHL-4 | (nM) | (nM) | (nM) | (%) |
| COMPOUND A | 743 | 14663 | 14663 | 1083 |
| ABT-199 | 673 | 12485 | 13046 | 316 |
| ABT99/GSK591_1/1 | 179 | 3251 | 3653 | −100 |
| fold change from PRMT5i | 4.2 | 4.5 | 4.0 | |
| fold change from BCL-2i | 3.8 | 3.8 | 3.6 | |

TABLE 2d

| | gIC50 | gIC100 | dEC50 | Ymin-T0 |
|---|---|---|---|---|
| WSU-DLCL2 | (nM) | (nM) | (nM) | (%) |
| COMPOUND A | 1165 | 14663 | 14663 | 2474 |
| ABT-199 | 6915 | 14663 | 14663 | 3182 |
| ABT99/GSK591_1/1 | 395 | 4863 | 5211 | −100 |
| fold change from PRMT5i | 3.0 | 3.0 | 2.8 | |
| fold change from BCL-2i | 17.5 | 3.0 | 2.8 | |

TABLE 2e

| | gIC50 | gIC100 | dEC50 | Ymin-T0 |
|---|---|---|---|---|
| WSU-FSCCL | (nM) | (nM) | (nM) | (%) |
| COMPOUND A | 388 | 14663 | 14663 | 224 |
| ABT-199 | 238 | 10530 | 14574 | −49 |
| ABT99/GSK591_1/1 | 91 | 909 | 1158 | −100 |
| fold change from PRMT5i | 4.2 | 16.1 | 12.7 | |
| fold change from BCL-2i | 2.6 | 11.6 | 12.6 | |

TABLE 2f

| | gIC50 | gIC100 | dEC50 | Ymin-T0 |
|---|---|---|---|---|
| OCI-LY3 | (nM) | (nM) | (nM) | (%) |
| COMPOUND A | 1964 | 14663 | 14663 | 488 |
| ABT-199 | 1466 | 7157 | 7508 | −76 |
| ABT99/GSK591_1/1 | 222 | 776 | 981 | −97 |
| fold change from PRMT5i | 8.9 | 18.9 | 14.9 | |
| fold change from BCL-2i | 6.6 | 9.2 | 7.7 | |

TABLE 2g

| | gIC50 | gIC100 | dEC50 | Ymin-T0 |
|---|---|---|---|---|
| U-2932 | (nM) | (nM) | (nM) | (%) |
| COMPOUND A | 281 | 1542 | 3067 | −88 |
| ABT-199 | 113 | 1366 | 3066 | −92 |
| ABT99/GSK591_1/1 | 29 | 108 | 181 | −100 |
| fold change from PRMT5i | 9.8 | 14.2 | 16.9 | |
| fold change from BCL-2i | 3.9 | 12.6 | 16.9 | |

TABLE 2h

| | gIC50 | gIC100 | dEC50 | Ymin-T0 |
|---|---|---|---|---|
| NU-DUL-1 | (nM) | (nM) | (nM) | (%) |
| COMPOUND A | 3728 | 12659 | 14663 | −11 |
| ABT-199 | 3021 | 4832 | | −45 |
| ABT99/GSK591_1/1 | 429 | 3981 | 7293 | −100 |
| fold change from PRMT5i | 8.7 | 3.2 | | |
| fold change from BCL-2i | 7.0 | 1.2 | 2.0 | |

TABLE 2i

| | gIC50 | gIC100 | dEC50 | Ymin-T0 |
|---|---|---|---|---|
| JV-M2* | (nM) | (nM) | (nM) | (%) |
| COMPOUND A | 139 | 2211 | | −14 |
| ABT-199 | 27 | 1607 | 9080 | −63 |
| ABT99/GSK591_1/1 | 9 | 92 | 380 | −100 |
| fold change from PRMT5i | 15.3 | 24.0 | | |
| fold change from BCL-2i | 3.0 | 17.4 | 24 | |

### Z-138 as Control for Compound A

TABLE 2j

| | gIC50 | gIC100 | dEC50 | Ymin-T0 |
|---|---|---|---|---|
| Z-138 | (nM) | (nM) | (nM) | (%) |
| COMPOUND A | 38 | 453 | 794 | −93.1 |
| ABT-199 | 14 | 1119 | 3123 | −78 |
| ABT99/GSK591_1/1 | 9 | 60 | 93 | −97 |
| fold change from PRMT5i | 4.1 | 7.5 | 8.6 | |
| fold change from BCL-2i | 1.6 | 18.6 | 33.7 | |

TABLE 3

| | gIC50 | gIC100 | dEC50 |
|---|---|---|---|
| ABT-199/Compound A | (nM) | (nM) | (nM) |
| OCI-LY3 | 7 | 9 | 8 |
| U-2932 | 4 | 13 | 17 |
| NU-DUL-1 | 7 | | |
| DB | 4 | 6 | 4 |
| SU-DHL-4 | 4 | 4 | 4 |
| WSU-DLCL2 | 3.0 | 3.0 | |
| WSU-NHL | | 3.1 | |
| DOHH-2 | | 5 | 5 |
| NALH-6 | | 4 | 6 |
| WSU-FSCCL | | 12 | 13 |
| JVM2 | 3 | 17 | 24 |

### Serial Dilutions

Serial dilutions were performed for the combination to ascertain the change in gIC₅₀, gIC₁₀₀ and dEC50 when the inhibitors are used together in a PRMT5 inhibitor cell proliferation. A first compound plate was prepared where PRMT5 inhibitor, Compound A, was prepared with a 20-point, two-fold dilution series of compound A from column 1 to 20. A second compound plate was prepared using the BCL-2 inhibitor ABT-199. ABT-199 was titrated in a 16-point 2-fold dilution series from row A to Q. A third plate was then created by added equal volume of 12 ul (ABT-199) from each well in the second plate into the corresponding well in the first plate. The third plate comprised rows from A to Q, each row comprising a fixed concentration of ABT-199. Columns 1-20 comprised varying concentrations of compound A from the 20-point dilution. Plates were incubated for six days at the conditions described above. Lymphoma cell line DB was treated with the solution in each of the wells. Cell growth was measured using CellTiter-Glo (Promega) and luminescence signal was detected with a BioTek Synergy Neo microplate reader. A plate of untreated cells was read at the time of compound/biologic addition to determine the T₀ value representing the starting number of cells. Data were fit with a four-parameter equation to generate a concentration response curve for each row using Assay Client software. A comparison was made across each row in the third plate to determine, when keeping a fixed concentration of ABT-199, what concentration of compound A is needed to reach the gIC₅₀, gIC₁₀₀ or dEC50 respectively and the fold change compared to treating the same cell with compound A alone.

Tables 4a-c show the results of the above serial dilution experiments wherein ‘fold change’ is the difference between the amount of compound A needed to achieve gIC₅₀, gIC₁₀₀ or dEC50 respectively, compared to the amount of compound A needed to achieve gIC₅₀, gIC₁₀₀ or dEC50 respectively when compound A is given as a single agent.

The concentration of ABT-199 at which ≥3 fold change was achieved in gIC₅₀, gIC₁₀₀ and dEC50 was 115 nM, 458 nM and 1833 nM respectively.

TABLE 4a

| | | Fold change over |
|---|---|---|
| | | compound A single |
| | compound A | agent gIC₅₀ (631 |
| | required for | nM, most potent |
| ABT-199 (nM) | gIC₅₀ (nM) | of the two agents) |
| 7331 | 15 | 43 |
| 3666 | 80 | 7.8 |
| 1833 | 74 | 8.5 |
| 916 | 78 | 8.1 |
| 458 | 119 | 5.3 |
| 229 | 115 | 5.5 |
| 115 | 209 | 3.0 |
| 57 | 247 | 2.6 |
| 29 | 351 | 1.8 |
| 14 | 387 | 1.6 |
| 7 | 449 | 1.4 |
| 4 | 514 | 1.2 |
| 2 | 509 | 1.2 |
| 1 | 445 | 1.4 |
| PRMT5 | 631 nM | 1.0 |
| inhibitor gIC₅₀ | | |
| ABT-199, | 6413 nM | NA |
| gIC₅₀ | | |

TABLE 4b

| | | Fold change over |
|---|---|---|
| | | compound A single |
| | compound A | agent gIC₁₀₀ (6489 |
| ABT-199 | required for | nM, most potent of |
| fixed, nM | gIC₁₀₀ (nM) | the two agents) |
| 7331 | 1707 | 3.8 |
| 3666 | 1745 | 3.7 |
| 1833 | 1604 | 4.0 |
| 916 | 1994 | 3.3 |
| 458 | 2118 | 3.1 |
| 229 | 2624 | 2.5 |
| 115 | 2716 | 2.4 |
| 57 | 3004 | 2.2 |
| 29 | 5525 | 1.2 |
| 14 | 6896 | 0.9 |
| 7 | 6362 | 1.0 |
| 4 | 6989 | 0.9 |
| 2 | 7331 | 0.9 |
| 1 | 7331 | 0.9 |
| PRMT5 single | 6489 | 1.0 |
| ABT-199, | 7331 | NA |
| single | | |

TABLE 4c

| | | Fold change over |
|---|---|---|
| | | top concentration |
| | | of compound A or |
| | | ABT-199 inhibitor |
| | | used in the assay |
| | Compound A | (since no dEC₅₀ |
| ABT-199 | required for | observed with |
| fixed, nM | dEC₅₀ (nM) | single agents) |
| 7331 | 2295 | 3.2 |
| 3666 | 2250 | 3.3 |
| 1833 | 2392 | 3.1 |
| 916 | 2528 | 2.6 |
| 458 | 3921 | 1.7 |
| 229 | 4657 | 1.4 |
| 115 | 4659 | 1.4 |
| 57 | 5370 | 1.2 |
| 29 | >7331 | NA |
| 14 | >7331 | NA |
| 7 | >7331 | NA |
| 4 | >7331 | NA |
| 2 | >7331 | NA |
| 1 | >7331 | NA |
| PRMT5 single | >7331 | NA |
| ABT-199, | >7331 | NA |
| single | | |

It will be understood that the present invention has been described above purely by way of example, and modification of detail can be made within the scope of the invention. Each feature disclosed in the description, and where appropriate the claims and drawings may be provided independently or in any appropriate combination.

1. A combination of a protein arginine methyltransferase 5 (PRMT5) inhibitor and a B cell lymphoma 2 (BCL-2) inhibitor.

2. The combination according to claim 1, wherein the PRMT5 inhibitor is a compound of Formula I: 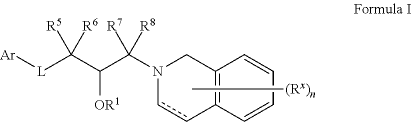
or a pharmaceutically acceptable salt thereof,
wherein [symbol]
: represents a single or double bond;
R¹ is hydrogen, R^{z}, or —C(O)R^{z}, wherein R^{z} is optionally substituted C₁₋₆ alkyl;
L is —N(R)C(O)—, —C(O)N(R)—, —N(R)C(O)N(R)—, —N(R)C(O)O—, or —OC(O)N(R)—;
each R is independently hydrogen or optionally substituted C₁₋₆ aliphatic;
Ar is a monocyclic or bicyclic aromatic ring having 0-4 heteroatoms independently selected from nitrogen, oxygen, and sulfur, wherein Ar is substituted with 0, 1, 2, 3, 4, or 5 R^{y} groups, as valency permits;
each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NRBC(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;
each R^{A} is independently selected from the group consisting of hydrogen, optionally substituted aliphatic, optionally substituted carbocyclyl, optionally substituted heterocyclyl, optionally substituted aryl, and optionally substituted heteroaryl;
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;
R⁵, R⁶, R⁷, and R⁸ are independently hydrogen, halo, or optionally substituted aliphatic;
each R^{X} is independently selected from the group consisting of halo, —CN, optionally substituted aliphatic, —OR′, and —N(R″)₂;
R is hydrogen or optionally substituted aliphatic;
each R″ is independently hydrogen or optionally substituted aliphatic, or two R″ are taken together with their intervening atoms to form a heterocyclic ring; and
n is 0, 1, 2, 3, 4, 5, 6, 7, 8, 9, or 10, as valency permits.

3. The combination according to claim 2, wherein the PRMT5 inhibitor is a compound of Formula II 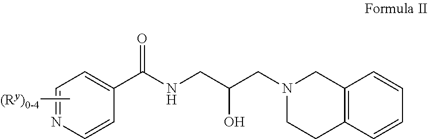
or a pharmaceutically acceptable salt thereof.

4. The combination according to claim 3, wherein the PRMT5 inhibitor is Compound A 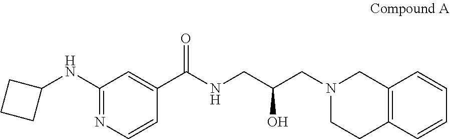 [SMILES: O=C(NC[C@H](O)CN1CCc2ccccc2C1)c1ccnc(NC2CCC2)c1]
or a pharmaceutically acceptable salt thereof.

5. The combination according to claim 2, wherein the PRMT5 inhibitor is a compound of Formula III 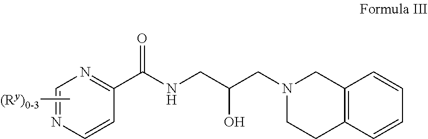
or a pharmaceutically acceptable salt thereof.

6. The combination according to claim 5, wherein the PRMT5 inhibitor is Compound B 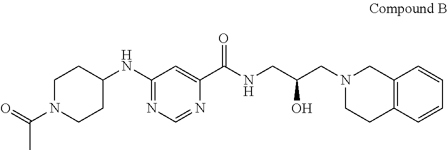 [SMILES: CC(=O)N1CCC(Nc2cc(C(=O)NC[C@H](O)CN3CCc4ccccc4C3)ncn2)CC1]
or a pharmaceutically acceptable salt thereof.

7. The combination according to claim 1, wherein the BCL-2 inhibitor is venetoclax (ABT-199), ABT-737, navitoclax (ABT-263), APG-1252, S-055746, BDA-366, HA14-1, BH3I-1, apogossypol, TW-37, TM12-06 or obatoclax.

8. The combination according to claim 7, wherein the BCL-2 inhibitor is venetoclax, ABT-737, navitoclax, APG-1252, obatoclax.

9. The combination according to claim 8, wherein the BCL-2 inhibitor is venetoclax.

10. A method of treating cancer in a human in need thereof, the method comprising administering to the human a combination according to claim 1.

11. The method according to claim 10, wherein the PRMT5 inhibitor and the BCL-2 inhibitor are administered to the patient simultaneously or sequentially.

12. The method according to claim 10, wherein the cancer is lung cancer or lymphoma.

13. The method according to claim 12, wherein the cancer is lymphoma.

14. The method according to claim 13, wherein the cancer is non-Hodgkin lymphoma.

15-19. (canceled)

20. A pharmaceutical composition comprising the combination of claim 1 and a pharmaceutically acceptable carrier.

21. A kit comprising a protein arginine methyltransferase 5 (PRMT5) inhibitor and a B cell lymphoma 2 (BCL-2) inhibitor according to claim 1.
